(19) United States
(12) Patent Application Publication
(10) Pub. No.: US 2016/0199492 A1
(43) Pub. Date: Jul. 14, 2016

(54) DIFFUSION PIGMENTS IN PHOTOTHERAPY

(71) Applicant: Merck Patent GmbH, Darmstadt (DE)

(72) Inventor: Corinna WIRTH, Darmstadt (DE)

(21) Appl. No.: 14/912,714

(22) PCT Filed: Jul. 18, 2014
(86) PCT No.: PCT/EP2014/001968

§ 371 Date: Feb. 18, 2016

(30) Foreign Application Priority Data
Aug. 22, 2013 (EP) 13004143.7

(51) Int. Cl. A61K 41/00; A61K 33/26; A61Q 19/02; A61N 5/06; A61K 8/25

(52) U.S. Cl. CPC A61K 41/0057; A61N 5/0616; A61N 5/0618; A61K 8/25; A61Q 19/02; A61K 33/26; A61N 2005/0654; A61N 2005/0655; A61N 2005/0653; A61N 2005/067

(57) ABSTRACT

The present invention relates to the use of microfine pigments in medical and cosmetic phototherapy.

The present invention relates to the use of microfine pigments for improving the light yield in phototherapy.

Phototherapy is one option of physically treating the skin. In it, the health-promoting properties of light of different wavelength are utilized. Phototherapy is used both in medicine and in cosmetics.

Many factors such as e.g. smoking, stress, sun, alcohol, air pollution and illnesses make the skin age prematurely and cause wrinkles. Such changes can also be attributed to natural ageing processes of the skin.

Targeted phototherapy in cosmetics revitalizes the skin and supports a healthy and radiant appearance. Light with specific wavelengths, which is generated for example by light emitting diodes (LEDs) or organic electroluminescent devices, such as for example organic light-emitting diodes (OLEDs) or organic light-emitting electrochemical cells (OLECs, LECs or LEECs), stimulates the production of collagen in the cells and activates cell growth. The increased formation of collagen results in wrinkles being reduced and the skin appearing more youthful, healthier and more vital. At the same time, targeted phototherapy inhibits enzymes which break down collagen and thereby increase the rate of the ageing process. Complete treatment in most cases lasts only a few minutes, is pain-free and is often even perceived as being pleasant. Typical cosmetic areas of application here are the treatment or prevention of skin ageing (anti-ageing), skin wrinkles, acne, comedones and cellulites.

Besides the cosmetic areas of application for phototherapy, there also exists a large number of medical or therapeutic areas of application. Thus, psoriasis, atopic dermatitis, atopic eczema, vitiligo, jaundice, newborn jaundice, itching of the skin, vitiligo, and many other inflammatory disorders of the skin are treated phototherapeutically. However, phototherapy is also used widely in pain therapy or in the treatment of tumors and in cancer therapy.

Besides the visible expression lines, the skin has a distinct microwrinkle structure. This gives rise to the typical skin relief with more or less distinct furrows. The result of this is that the actual surface of the skin is considerably larger than the planar projection portrays. However, the result of this is also that during a phototherapeutic treatment of the skin, the areas of skin lying in the furrows are irradiated less intensively than the areas which are outside of the furrows on the surface of the skin. During a phototherapeutic treatment, this leads to the areas within the furrows not being optimally irradiated. Or else, in cases where the areas of skin in the furrows are sufficiently irradiated, it leads to the areas outside of the furrows on the surface of the skin being irradiated too intensively. As a result, the nature of the skin hinders a uniform illumination of the entire available surface. It is often necessary to administer higher light doses, which may lead to increased side effects due to the phototherapy. This considerably limits phototherapy possibilities.

Proceeding from this, it is the object of the present invention to improve the cosmetic and medical phototherapeutic treatment in such a way that uniform illumination of the entire skin is achieved.

The present object is achieved by the features of claim 1. Further advantageous embodiments of the invention are the subject of the dependent claims.

The present invention achieves the stated object by virtue of the fact that the pigments used in the phototherapy include diffusion pigments. Through the use according to the invention of diffusion pigments, a considerable improvement in the phototherapy is achieved by avoiding an overloading of the illuminated area and ensuring optimum biological utilization of the light. By virtue of the technical teaching according to the invention it is possible to use lower light intensities in order to achieve the same effect with fewer side effects, or else it is possible to achieve a higher effect of the phototherapy using identical light intensities. The higher light intensities can, on the one hand, be used to increase the effect of radiation at the site of action. On the other hand, the higher light intensities can also be utilized at the site of action in order to reduce the irradiation time.

Diffusion pigments are included among the cosmetic fillers and are notable for the fact that they have an irregular surface. The irregular surface results in incident light being irregularly reflected, and thus diffused, i.e. scattered. This effect is also referred to as “Soft Focus Effect”. Diffusion pigments with these effects are sometimes also referred to as “Soft Focus Fillers”.

A typical example of a diffusion pigment (Soft Focus Filler) is the pigment Ronasphere® LDP from Merck KGaA. Ronasphere® LDP is a functional filler which consists of silica beads coated unevenly with titanium dioxide and iron oxide. As a result of this coating, the beads have a light-diffusing, and virtually nonreflective effect. Their refractive index is close to that of skin. If they are applied to the skin, they are virtually invisible on account of the lack of reflective effect. The light diffusion properties of Ronasphere® LDP permit the formulation of skincare mixtures and decorative cosmetics with special optical effects.

The light-diffusing properties contribute to optically correcting fine lines and wrinkles. These microbeads are also used as a galenical auxiliary since, in aqueous compositions, they help to increase the viscosity, as a result of which the use of additional thickeners or gelling auxiliaries can be avoided. With gentle stirring, the particles can easily be dispersed in water or oil. 80% of the Ronasphere® LDP particles have a particle size of less than 25 micrometers; the average size is 4.0 to 7.0 micrometers.

Formulations based on silicones are often notable for a very specific, elegant-pleasant skin feel, but cannot always be fully realized in formulations for reasons of cost. A sensory approximation in this direction is possible in these cases also through targeted use of spherical pigments. The Soft Focus Effect is best supported through the use of bead-shaped substances.

As a result of the use according to the invention of the diffusion pigments, the photons that are available are utilized more efficiently. Surprisingly, it was able to be shown that the diffusion pigments used not only scatter daylight, and as a result make skin lines appear softer, but also distribute the light used in the course of a phototherapeutic treatment of the skin, or the radiation from an artificial irradiation source in such a way that the skin of the areas in the furrows are irradiated more intensively compared with the prior art than the areas which are outside of the furrows on the surface of the skin. During a phototherapeutic treatment, this leads to the result of all of the areas of the skin being sufficiently irradiated.

The light intensity of phototherapy can accordingly be reduced, which has a positive effect on treatment comfort due to a shortened irradiation time or a lower operating temperature of the illumination device.

Furthermore, the use of the Soft-Focus Filler allows light sources to be used for the phototherapy which have hitherto not been suitable for this purpose on account of their low radiation intensity.

Similarly, the heating effect of the light (for example IR radiation) can be utilized more efficiently since increased heat transmission into the skin and thus the tissue is brought about by the use according to the invention of the diffusion pigments.

In principle, any radiation source can be used for the purpose of phototherapy. It is preferred if an artificial radiation source is used for the phototherapy. Typical artificial radiation sources which are used in phototherapy are conventional lamps, UV lamps, fluorescent tubes, lasers or LEDs. A further class of radiation sources is the organic electroluminescent devices. The organic electroluminescent devices include in particular also the organic light-emitting diodes (OLED), the organic light-emitting transistors, the organic light-emitting electrochemical cells (OLEC, LEC or else LEEC) and the organic laser diodes. Among the organic electroluminescent devices, the OLEDs and OLECs are particularly preferred in the context of the present invention, and the OLEDs are very particularly preferred.

A person skilled in the art will encounter no difficulties at all in selecting the radiation source suitable for the respective purpose from a large repertoire of radiation sources. For example, for stationary applications, which are preferably carried out in medical or cosmetic installations, conventional lamps, UV lamps, fluorescent tubes, lasers or LEDs are often used. For private use or mobile use, organic electroluminescent devices in particular are suitable on account of their small size and flexibility of the devices. However, organic electroluminescent devices are also suitable for stationary areas of application.

Furthermore, the person skilled in the art faces no difficulties at all in selecting the appropriate wavelength for the particular phototherapeutic application. In this regard, there are numerous publications which recommend specific wavelengths or ranges of wavelength for phototherapeutic treatment and which have also in the meantime proven successful in the therapy.

It is preferred in the context of the present invention if the radiation source emits radiation with a wavelength in the range from 250 nm to 1500 nm.

For the treatment of acne, blue light is very preferred. It is particularly preferred if the radiation source emits blue light of wavelength 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429 and 430 nm in order to treat acne. 414 and 415 nm are of particular interest in order to kill off P. Acnes bacteria.

Treatment in this connection is understood as meaning both treatment of existing conditions (e.g. acne), and also prophylaxis.

Studies relating to the treatment of acne have revealed that its combination of different wavelengths are very particularly suited to effectively treating acne. In this connection, a combination of blue and red light is particularly preferred. The red light is preferably selected here from the wavelength range from 590 to 750 nm, very preferably from the range from 600 to 720 nm and very particularly preferably from the range from 620 to 700 nm. The wavelengths 633 and 660 nm are particularly preferred. The blue light can be selected from the aforementioned wavelengths.

For the treatment of comedones, a wavelength of 500 nm or light in the range from 500 to 700 nm is preferred.

For the treatment of cellulite, preference is given to using a wavelength in the range from 400 to 1000 nm, very preferably in the range from 400 to 900 nm, particularly preferably in the range from 450 to 900 nm and very particularly preferably in the range from 500 to 850 nm.

For the treatment of skin ageing, preference is given to using a wavelength in the range from 400 to 950 nm, very preferably in the range from 550 to 900 nm and particularly preferably in the range from 550 to 860.

Furthermore, for the treatment of skin ageing in a preferred embodiment of the present invention, wavelengths in the range from 600 nm to 650 nm, very preferably in the range from 620 nm to 650 nm, is used.

Furthermore, it is preferred for the treatment of skin ageing if the radiation source emits light radiation in the range from 350 to 950 nm, very preferably in the range from 380 to 900 nm and particularly preferably in the range from 400 to 900 nm.

Blue light is particularly preferred for the purpose of treating skin ageing.

Further very particularly preferred wavelengths for the treatment of skin ageing are 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, and 430 nm. 415 nm is a particularly preferred wavelength here.

For the treatment of dermatitis, preference is given to using a wavelength in the range from 470 to 670 nm, very preferably in the range from 490 to 650 nm and particularly preferably in the range from 530 to 610 nm. Two particularly preferred wavelengths for the treatment of dermatitis are 550 nm and 590 nm.

For the treatment of atopic eczema, preference is given to using a wavelength in the range from 470 to 670 nm, very preferably in the range from 490 to 650 nm and particularly preferably in the range from 530 to 610 nm. A very particularly preferred wavelength in this connection is 320 nm.

For the treatment of psoriasis, preference is given to using a wavelength in the range from 240 to 500 nm, very preferably in the range from 290 to 400 nm and particularly preferably in the range from 300 to 330 nm. Two very particularly preferred wavelengths in this connection are 311 nm and 320 nm.

For the treatment of vitiligo, preference is given to using a wavelength in the range from 240 to 500 nm, very preferably in the range from 290 to 400 nm and particularly preferably in the range from 300 to 330 nm. A very particularly preferred wavelength in this connection is 311 nm.

For the treatment of edemas, preference is given to using a wavelength in the range from 760 to 940 nm, very preferably in the range from 780 to 920 nm, particularly preferably in the range from 800 to 900 nm and very particularly preferably in the range from 820 to 880 nm. A very particularly preferred wavelength in this connection is 850 nm.

For the treatment of inflammations, preference is given to using a wavelength in the range from 350 to 900 nm, very preferably in the range from 380 to 900 nm and particularly preferably in the range from 400 to 860 nm. Very particularly preferred wavelengths in this connection are 405 nm, 420 nm and 850 nm.

For the treatment of alopecia, preference is given to using a wavelength in the range from 240 to 500 nm, very preferably in the range from 290 to 400 nm and particularly preferably in the range from 300 to 330 nm, A very particularly preferred wavelength in this connection is 311 nm.

For the treatment of jaundice and neonatal jaundice, preference is given to using a wavelength in the range from 300 to 700 nm, very preferably in the range from 350 to 600 nm, particularly preferably in the range from 370 to 580 nm, very particularly preferably in the range from 400 to 550 nm and particularly preferably in the range from 410 to 470 nm. Two very particularly preferred wavelengths in this connection are 450 and 466 nm.

For some applications, it may be advantageous if the phototherapeutic irradiation of the skin takes place continuously, whereas in other applications a pulsed irradiation is desirable. The person skilled in the art can find typical pulse sequences in the prior art. WO 2003/086215 A1 describes, for example in example 1, preferred pulse sequences for reducing skin wrinkles.

In a preferred embodiment, the phototherapy takes place by means of organic electroluminescent devices, preferably by means of OLEDs and/or OLECs. An advantage of organic electroluminescent devices is that they are thin-layer structural elements made of organic materials which, in contrast to LEDs, have a lower current density, have a lower luminance and require no monocrystalline materials. The organic electroluminescent devices can be produced on flexible supports, are planar emitters and can be produced in any desired fitting shape for the body parts to be irradiated. Details on the specified devices are well known in the prior art. The wavelengths of the specified devices can be adapted to the particular cosmetic or medical requirements by the person skilled in the art through suitable selection of fluorescent or phosphorescent emitters or color converters without exercising inventive skill. One advantage of OLECs over OLEDs consists in the simpler construction and the use of fewer materials that are harmful to health. OLECs can mostly be produced more cost-effectively and, on account of the mostly shorter service life compared with the service life of the OLEDs, are suitable in particular for short-lived products which are used only once or a few times, such as, for example, plasters. The input of energy for the organic electroluminescent devices can take place by means of a mains adapter, but also by means of its own power supply using a small battery or in the form of a printed battery. A typical construction of an OLED is disclosed in WO 90/13148 A1. Further details relating to OLEDs can also be found in J. Shinar, Organic Light-Emitting Devices, Springer Verlag 2004. OLECs are disclosed, for example, by Qibing Pei et al. in Science, 1995, 269, 1086-1088 and in WO 2012/045384 A1.

The properties of a diffusion pigment (Soft Focus Filler) can clearly be described by the Soft Focus Factor (SFF). The SFF depends on two essential optical properties, the degree of whiteness and the matting effect.

The quantification of SFF is described in detail in WO 2012/031665 A1 by reference to a goniochromatic measurement method. The SFF values given presently are ascertained by means of this method. SFF is accordingly defined as the quotient of Lw and Lg (SFF=Lw/Lg), where Lw is the lightness value L* at 65° and Lg is the L* value at the glancing angle. Further details relating to the parameters and the implementation of the measurement method can be found in the disclosure of WO 2012/031665 A1. Ideally, the Soft Focus Factor is 1.

It is preferred in the context of the present invention if the diffusion pigment used according to the invention has an SFF in the range from 0.5 to 1, very preferably in the range from 0.6 to 1, particularly preferably in the range from 0.65 to 1, very particularly preferably from 0.7 to 1, especially preferably in the range from 0.75 to 1, even more preferably in the range from 0.8 to 1 and most preferably in the range from 0.85 to 1.

It is of particular advantage if the diffusion pigments comprise at least one substance which is selected from the group of the following substances: Powder preparations of siliceous earth and silicates, talc, powder preparations of polyethylenes, powder preparations of starch, powder preparations of polyamides, powder preparations of styrenes and/or acrylic copolymers, silicone elastomers, aluminum silicate powder, powder preparations of the polymethyl methacrylate (PMMA) type, talc, compositions of siliceous earth and TiO₂ or siliceous earth and zinc oxide, polyethylene powder, starch powder, polyamide powder, compositions of styrene and acrylic copolymers, silicone elastomers, and mixtures thereof.

The following compounds, for example, can also be used: Talc with an average size of less than 3 μm, talc with an average size of less than 1.8 μm, TALC P3.® from Nippon Talc; NYLON.® 12 powder, ORGASOL 2002 EXTRA D NAT COS.® from Atochem, siliceous earth particles whose surface has been treated with 1 to 2% mineral wax, for example from Degussa, amorphous siliceous earth microspheres such as, for example, of the type SUNSPEHRE (reference H-53) from Asahi Glass, and microbeads such as SB-700.® or SB-150.® from Miyoshi.

A composition for the phototherapy that has proven to be advantageous for the cosmetic or medical application is one which comprises at least one cosmetic base or one drug base, and diffusion pigments. Depending on the nature of the skin and the aim of the cosmetic or medical treatment, different fractions of diffusion pigments can be used.

The preparation of the compositions is well known to the person skilled in the art from the prior art. Examples of corresponding preparations are disclosed in WO 00/15720 A1, WO 01/16235 A1 or in WO 2012/031 664 A1, as well as in the examples.

In a further preferred embodiment of the present invention, the cosmetic composition comprises at least one cosmetic active ingredient. As a result of this, the effect of the phototherapeutic radiation can be further increased.

In a further preferred embodiment of the present invention, the pharmaceutical composition comprises at least one pharmaceutical active ingredient. As a result of this, the effect of the phototherapeutic radiation can be further increased.

Moreover, it has proven to be advantageous if a composition at least comprising one diffusion pigment is used as drug.

The cosmetic or medical phototherapy can be further improved by treating the skin to be irradiated with skin-lightening compounds. Skin-lightening compound is understood as meaning all compounds which are understood as such according to WO 2013/060407 A1. The treatment of the skin with a skin-lightening compound is preferably carried out prior to the phototherapeutic treatment of the skin with the diffusion pigment, but can also take place at the same time as this. The treatment of the skin with a skin-lightening compound has the technical effect that more radiation from the radiation source can penetrate into the layers of the skin. The technical effect according to the invention brought about by the use of diffusion pigments in the phototherapy can thus be further increased through the additional use of skin-lightening compounds. Further details relating to the use of the skin-lightening compounds can be found in the disclosure of WO 2013/060407 A1.

The combined application of diffusion pigments and skin-lightening compounds in the phototherapy is particularly suitable for the treatment of jaundice, preferably of neonatal jaundice in newborns with skin type II to VI, very preferably with skin type IV to VI and particularly preferably with skin type V to VI (WO 2013/060407 A1).

The present invention therefore also relates to a composition comprising at least one diffusion pigment and at least one skin-lightening compound.

The present invention also relates to a method for the cosmetic or medical treatment of human or animal skin, comprising the steps 1) Treatment of the skin with a skin-lightening compound, 2) Treatment of the skin with a diffusion pigment, and 3) Irradiation of the skin with a radiation source.

It is known to the person skilled in the art that the skin-lightening compounds, as well as the diffusion pigments in general are applied with the help of corresponding medical or cosmetic compositions.

The present invention furthermore relates to a kit consisting of a composition comprising at least one diffusion pigment and a composition comprising at least one skin-lightening compound.

In a preferred embodiment, the kit also comprises an artificial radiation source which is very preferably selected from the aforementioned radiation sources.

It is of particular advantage here if the treatment of skin diseases takes place by means of phototherapy.

A particularly advantageous cosmetic method for improving the skin has proven to be one which involves the steps preparation of the skin, application of a composition comprising diffusion pigments and irradiation of the skin by means of light.

It is particularly advantageous here if in the cosmetic process the irradiation takes place by means of OLED and/or OLEC.

The technical teaching according to the invention is characterized by one or more of the following surprising advantages over the prior art:
1. The technical teaching according to the invention permits, while retaining the effect, the light intensity emitted by the radiation source to be reduced for a phototherapeutic application in medicine and/or cosmetics. Side effects of phototherapeutic applications can be reduced.
2. As a result of the fact that lower radiation intensities are required, the use of modern radiation sources, such as that of the organic electroluminescent devices, is further driven forward.
3. As a result of the fact that lower radiation intensities are required, energy can be saved.
4. The technical teaching according to the invention permits the use of mobile radiation sources with a low energy demand.
5. The technical teaching according to the invention permits the radiation intensity and thus the effect at the site of action to be increased without increasing the intensity of the radiation emitted by the radiation source.
6. By virtue of the higher radiation intensity attained at the site of action of the radiation, it is possible to reduce the irradiation time, which has a positive effect on the reduction of side effects and signifies greater comfort for the person to be irradiated.

These abovementioned advantages are not accompanied by a deterioration in the further electronic properties.

The abovementioned preferred embodiments can be combined with one another as desired. In a particularly preferred embodiment of the invention, the abovementioned preferred embodiments apply simultaneously.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

It should also be pointed out that many of the features, and especially those of the preferred embodiments of the present invention, are themselves inventive and should not be regarded merely as some of the embodiments of the present invention. For these features, independent protection may be sought in addition to or as an alternative to any currently claimed invention.

The technical teaching disclosed with the present invention may be abstracted and combined with other examples.

The person skilled in the art can develop further phototherapeutic applications according to the invention from the descriptions without exercising inventive skill and thereby implement the invention over the entire claimed range.

The examples and drawings below describe embodiments of the invention by way of example. These examples serve to illustrate the invention and are not to be understood in a limiting sense.

FIG. 1 shows the transmission in % as a function of the wavelength in nm of treated and untreated PMMA plates. The top line (solid) shows the values for untreated PMMA plates (PMMA_1), the bottom line (dotted) shows the data of PMMA plates treated with Ronaflair® LDP white (LDP_1).

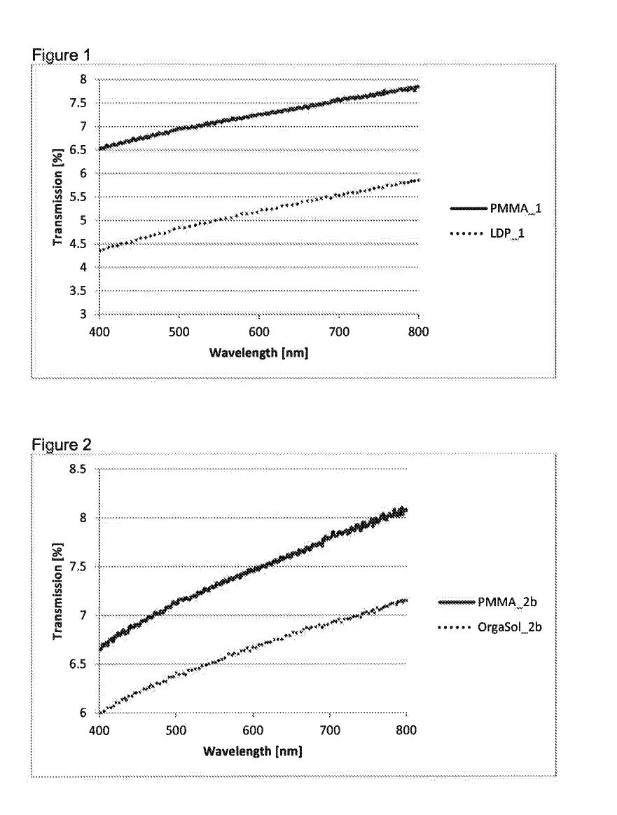

FIG. 2 shows the transmission in % as a function of the wavelength in nm of treated and untreated PMMA plates. The top line (solid) shows the values for untreated PMMA plates, the bottom line (dotted) shows the data of PMMA plates treated with Orgasol.

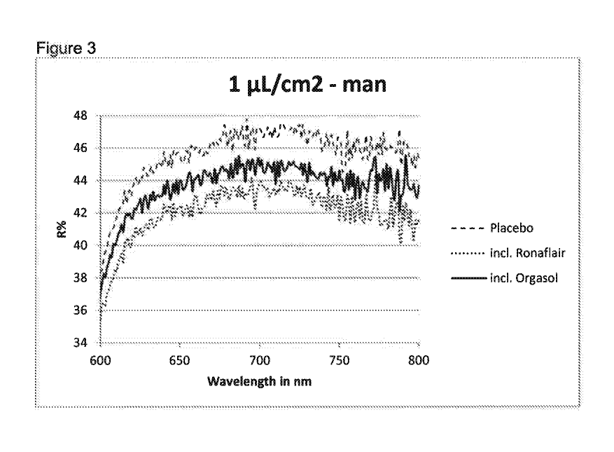

FIG. 3 shows the remission in % as a function of the wavelength in nm. The top line at 700 nm shows the values for placebo (dashed), the middle line (solid) shows the values for Orgasol and the bottom line (dotted) shows those for Ronaflair®.

### EXAMPLES

### Example 1

### In Vivo Test: Heat Input Into the Skin

Two standard commercial red light lamps (Philips HP 3616/01 INFRAPHIL) are mounted in a fixed manner in each case at a height of 25 cm above the backs of the hand of a volunteer placed on a table. In parallel, the temperature perception of the one hand A (as control treated with base cream) is then compared with the other B (treated with a base cream comprising 8% Ronaflair® LDP white (Merck KGaA)). It was found that the temperature of the back of hand B is perceived as warmer. Formulations which comprise different diffusion pigments exhibit the same properties. Ronaflair® LDP white is a preparation of silica with a TiO₂/Fe₂O₃-coating.

### Example 2

Polymethyl methacrylate plates (1 mm thickness) are roughened in order to simulate a skin surface profile. Then, the transmission is measured between 400 and 800 nm in a spectrometer. For this, an untreated plate is compared as comparison and a plate coated with Ronaflair® LDP white (SFF=0.89). If was found (FIG. 1) that the direct transmission is reduced by the filter, the light rays is broken off by the radiation source, which the scattering of the incident light proves. The diffusion pigment itself is transparent and cannot explain the reduced transparency by light absorption.

The experiments are carried out with different diffusion pigments and exhibit a comparably good effect. FIG. 2 shows the effect when using OrgaSol (SFF=0.84). Analogous good results are also attained with RonaFlair® ExtenderW (Merck KGaA; SFF=0.8), TRES BN PUHP3016 (Saint-Gobain; SFF=0.84) and Coverleaf AR80 (Presperse Inc.; SFF=0.82).

Conventional fillers (e.g.. RonaFlair® Micra M (Merck KGaA; SFF=0.15) or talc (MerckKGaA; SFF=0.21)) exhibit a considerably lesser ability to distribute incident light.

### Example 3

### In Vivo Experiment on Humans

Three identically sized areas are drawn onto the inside of the forearm of a male volunteer subject and treated with control formulation and two verum formulations in a defined application amount.

The amount of light radiated back from the surface of the skin is quantified by means of standard commercial emission measurement by means of a remission probe. The control used is a standard commercial formulation (Ombia Natursole Salbe), which has no particulate ingredients. In each case, 5% Orgasol or 5% Ronaflair® LDP white is added to this formulation. In each case 1 μL/cm² are applied.

It is found that the remission of the placebo formulation is greater than that of the verum formulations. Consequently, due to a reduced remissions, more light is available in the skin, which leads to a better biological efficacy (FIG. 3).

### Example 4

### Preparation of a Formulation Comprising Diffusion Pigments

### 4.1 Preparation of a Mixture Comprising Diffusion Pigments

122.7 g of spherical silicon dioxide (Ronaflair® LDP white) are suspended in 2277 g of demineralized water and the suspension is heated to 75° C. Then, 88.5 g of titanium tetrachloride solution (TiCl₄ content: 64.4% by weight) are metered in with 90 g of demineralized water over a period of 60 minutes. 32% strength sodium hydroxide solution is used to keep the pH constant at 2.2 to 2.3. Following the metered addition of the titanium tetrachloride solution, the pH is adjusted to 3.2. Then, 50.4 g of iron(III) chloride solution, which are diluted with 50.4 g of water, are added, the pH being held at 3.2 to 3.2. Then, the mixture is stirred for 15 minutes and the pH is adjusted to 7.0.

The suspension is left to cool and settle overnight. The solution above the sediment is decanted off and discarded. The sediment is isolated via a suction filter, washed salt-free with 5 L of demineralized water, dried overnight at 110° C. in a drying cabinet and passed through a 0.3 mm sieve. This gives 154 g of component B.

9.2 kg of spherical silicon dioxide (Ronaflair® LDP white) are suspended in 170 L of demineralized water and the suspension is heated to 75° C. with stirring. Then, 10.43 L of titanium tetrachloride solution are diluted with 3 L of demineralized water and added to the suspension over a period of 60 min. By adding 32% strength sodium hydroxide solution, the pH is kept constant at 2.2 to 2.3. Following the metered addition of the titanium tetrachloride solution, the mixture is stirred for a further 15 minutes and the pH is adjusted to 7.0. Component A prepared in this way is left in the reactor.

Then, 715 g of the dried and sieved component B are added to the suspension of component A in the reactor. The mixture is stirred for 15 min. and left to cool and settle overnight. After decanting off the supernatent solution, the sediment is filtered off via a single-layer filter and washed salt-free with 1000 L of demineralized water. The resulting pigment is dried overnight at 110° C. and calcined at 650° C. in a rotary kiln with a residence time of 15 minutes. This gives a wool-white powder with its very good skin feel.

### 4.2 Preparation of a Cosmetic Base

To produce a cosmetic base, in a first phase A, 7% by weight of caprylic/capric triglyceride, 4% by weight of refined jojoba wax, 6% by weight of ethylhexylene stearate, 2% by weight of glycerol monostearate, 2% by weight of Generol R, i.e. of the stated mixture of phytosterols, and 1.5% by weight of polyglycerol-3 methyl glucose distearate are prepared. 0.15% by weight of carboxypolymethylene, 0.15% by weight of acrylic acid vinyl ester copolymer, 4% by weight of 86.5% strength glycerol, 2% by weight of urea and 0.1% by weight of sodium hyaluronate are introduced into heated water with a weight fraction of 59.75% by weight. The resulting phase B is heated further and added to phase A again with gentle stirring.

As phase C, 5% by weight of SK-Influx, i.e. of a mixture of ceramides, carbomers and xanthan, is added to the resulting mixed phase of phases A and B with gentle stirring. Following homogenization of the resulting mixture, it is cooled to about 40° C.

### 4.3 Preparing the Preparation

Depending on the nature of the skin, 2 to 5% by weight of the mixture comprising diffusion pigments are then added to the cosmetic base; finally, the resulting preparation is homogenized.

Compositions for the pharmaceutical application can be prepared analogously. The present invention improves the options of phototherapeutic skin treatment in a decisive manner and thereby expands its cosmetic and medical possibilities.

1-18. (canceled)

19. A composition comprising a diffusion pigment having a soft focus factor in the range from 0.5 to 1

20. The composition of claim 19, wherein the diffusion pigment has a soft focus factor is in the range from 0.65 to 1.

21. The composition of claim 19, wherein the diffusion pigment has a soft focus factor is in the range from 0.8 to 1.

22. The composition of claim 19, comprising at least one powder preparation selected from a group consisting of siliceous earth and silicates, polymethyl methacrylates, talc, a mixture of siliceous earth and TiO₂, a mixture of siliceous earth and zinc oxide, polyethylenes, starch, polyamides, styrenes, acrylic copolymers, and silicone elastomers.

23. The composition of claim 19, further comprising a skin-lightening compound.

24. A process comprising applying the composition of claim 19 to the skin of a patient or animal in need of therapeutic treatment, and irradiating the applied composition with a radiation source.

25. The process of claim 24, wherein the patient is diagnosed with a disease state selected from the group consisting of psoriasis, atopic dermatitis, atopic eczema, jaundice, neonatal jaundice, vitiligo, edemas, tumors, inflammations, depression, pain and seasonal affective disorder.

26. The process of claim 24, wherein the radiation source is selected from the group consisting of a conventional lamp, UV lamp, laser, fluorescent tubes, LED, an organic electroluminescent device, and an organic electroluminescent cell.

27. The process of claim 24, wherein the radiation source is selected from an organic electroluminescent device, or an organic electroluminescent cell.

28. The process of claim 27, wherein the diffusion pigment has a soft focus factor is in the range from 0.65 to 1.

29. A process comprising applying the composition of claim 19 to the skin of a patient in need of treatment and prevention of a condition state selected from acne, cellulite, skin ageing, or skin wrinkles, and irradiating the applied composition with a radiation source.

30. The process of claim 29, wherein the radiation source is selected from an organic electroluminescent device, or an organic electroluminescent cell.

31. A process comprising applying the composition of claim 23 to the skin of a patient in need of treatment and prevention of a condition state selected from acne, cellulite, skin ageing, or skin wrinkles, and irradiating the applied composition with a radiation source.

32. The process of claim 30, wherein the diffusion pigment has a soft focus factor is in the range from 0.65 to 1.

33. A kit comprising a composition of claim 19 and at least one skin-lightening compound.

34. The kit of claim 33, further comprising a radiation source selected from the group consisting of a conventional lamp, UV lamp, laser, fluorescent tubes, LED, an organic electroluminescent device, and an organic electroluminescent cell.

35. A method of treating a patient diagnosed with one or more of the following disease states selected from the group consisting of psoriasis, atopic dermatitis, atopic eczema, jaundice, neonatal jaundice, vitiligo, edemas, tumors, inflammations, depression, pain and seasonal affective disorder, the method comprising applying a diffusion pigment having a soft focus factor in the range from 0.5 to 1, and irradiating the applied composition with a radiation source.

36. The method of claim 35, wherein the diffusion pigment has a soft focus factor is in the range from 0.65 to 1.

37. A method of treating a patient with acne, cellulite, skin ageing, or skin wrinkles, the method comprising applying a diffusion pigment having a soft focus factor in the range from 0.5 to 1, and irradiating the applied composition with a radiation source.
